(19) United States
(12) Patent Application Publication
(10) Pub. No.: US 2016/0124063 A1
(43) Pub. Date: May 5, 2016

(54) Magnetic Relaxometry to Assess Disease via Circulating Markers

(71) Applicant: Senior Scientific LLC, Albuquerque, NM (US)

(72) Inventor: V Gerald Grafe, Corrales, NM (US)

(21) Appl. No.: 14/861,053

(22) Filed: Sep. 22, 2015

Related U.S. Application Data
(60) Provisional application No. 62/072,293, filed on Oct. 29, 2014.

(51) Int. Cl. G01R 33/44; G01R 33/56; A61B 5/00; G01R 33/34; A61B 5/145; A61B 5/055

(52) U.S. Cl. CPC G01R 33/448; A61B 5/14546; A61B 5/055; A61B 5/6824; G01R 33/34023; G01R 33/5601

(57) ABSTRACT

Methods and apparatuses for detecting substances circulating in a patient's blood are described. The substances, such as tumor cells, are targeted with nanoparticles labeled such that they preferentially bind with the substance of interest. As the nanoparticle-labeled substance circulates, it can pass near a magnetic relaxometry system that can detect the substance by detecting the nanoparticles bound to it.

### CROSS-REFERENCE TO RELATED APPLICATIONS

This application claims priority to U.S. provisional 62/072,293, filed Oct. 29, 2014, which is incorporated herein by reference.

### DESCRIPTION OF INVENTION

Magnetic relaxometry has been proposed to locate and measure solid tumors and other disease with fixed sites in vivo. See, e.g., PCT/US2010/055729; PCT/US2011/028746; PCT/US2011/039349; U.S. Ser. No. 13/870,925 filed Apr. 25, 2013; U.S. Pat. No. 8,447,379 issued May 21, 2013; each of which is incorporated herein by reference. The present invention provides methods and apparatuses for assessing disease expressed in cells or other substances circulating in a body.

Superparamagnetic nanoparticles are conjugated with antibodies or other targeting agents that preferentially bind to a disease marker, using processes known in the art and processes like those described in the references mentioned elsewhere herein. Targeting agents can comprise, as examples, antibodies that are specific to markers such as cell surface antigens on cancer cells, antibody fragments, small molecules, or any other substance that will bind the nanoparticle to a marker for disease. The specificity of the targeting agent contributes to the specificity of the invention. For example, nanoparticles using her-2 antibodies as a targeting agent will bind with cells expressing her-2; while nanoparticles using an EGFR as a targeting agent will bind with cells expressing EGFR, generally encompassing a greater number of cells and wider range of cell types.

The targeted nanoparticles (nanoparticles conjugated with a targeting agent) are introduced into the circulatory system of a subject, either animal or human. While in the circulatory system, if they encounter a fixed tumor (or other site with receptors for the targeting agent), the targeting agent will bind to the receptors and the nanoparticle will be effectively removed from circulation (at least for a time). If the site to which the nanoparticle was bound breaks free, e.g., a cancer cell breaks free of the fixed tumor and enters the circulatory system, then the nanoparticle will re-enter the circulatory system attached to the much larger cell.

If a targeted nanoparticle encounters a targeted site in the circulatory system, e.g., by encountering a circulating tumor cell, then the nanoparticle will be bound to the circulating cell and accompany it through the circulatory system.

The presence in the circulatory system of targeted cells (or other substances with receptors for the specific targeting agent) can be an indicator of disease. As an example, cancer tumors can shed cells into the circulatory system. The presence of such cells can indicate the presence of a tumor. Such circulating tumor cells can also be indicators that a tumor might metastasize to another location.

A magnetic relaxometry measurement system can be placed in proximity to the circulatory system of the subject. As an example, such a system can be placed on a wrist next to veins in that region. Other sites near blood flow can also be suitable. Such a system can also be made indwelling, and be placed within an artery or vein. Such a system can also be placed in an extracorporeal blood loop. The system can be mounted semipermanently, as with a wristwatch; can be mounted with clothing, such as gloves; and can be configured as a separate measurement device that is used to make measurements only when a subject is nearby. Since magnetic relaxometry involves measuring magnetic fields, it is generally preferable to place the sensors close to the region to be measured. If frequent, or near-continuous, monitoring is desired, then mounting such as with a wristwatch, can be preferable. The measurement system can also comprise communications with a remote system for control of the measurement system, data collection from the measurement system, analysis of data from the measurement system, or a combination thereof. For example, a wrist-mounted measurement system can be placed in wired or wireless communication with a smart phone or computer, wherein the smart phone or computer controls the measurement system and analyses data from the measurement system.

A magnetic relaxometry system involves a magnetization facility and a sensor facility. The magnetic characteristics of the nanoparticles determine the timing of the measurement. It has been found that iron oxide nanoparticles of about 25 nm in diameter are suitable for a measurement with a 0.75 second magnetization pulse followed by a magnetic field measurement over about 3 seconds. The 3 second measurement time allows Neel relaxation of nanoparticles that have bound to relatively large objects (such as cells) to be distinguished from randomization of magnetic moments to Brownian motion of nanoparticles that have not bound to such objects.

The blood flow characteristics of the region to be sampled can determine the configuration of the magnetic relaxometry system. The magnetization system and sensor system can be separated by a distance such that nanoparticles magnetized by the magnetization system are within the measurement region of the measurement system during the appropriate time after magnetization. Using the example numbers above, the sensor system can be positioned downstream from the magnetization system a distance that nanoparticles will traverse in about 0.5 to 3 seconds.

In operation, the magnetic relaxometry system can detect when nanoparticles bound to the targeted substance are in the measurement region of the magnetic relaxometry system. Such detection can be used for various purposes, depending on the targeting agent. As examples: (a) If a specific tumor antigen is targeted, then the detection of such tumor cells by the system can indicate that a tumor of that type is present, and that further tests are indicated. (b) If a tumor is known or suspected, then nanoparticles targeting that tumor type can be used. The nanoparticles will be expected to bind to the solid tumor at the fixed site. If bound nanoparticles are subsequently detected by the system, then it can be inferred that the tumor is shedding cells and possibly metastasizing, possibly indicating that more aggressive treatment is indicated. (c) A targeting agent that targets markers that are common to many disease types, e.g., EGFR or VEGF, can be used. The detection of those cells by the system can indicate that more specific tests are needed. (d) A plurality of targeting agents can be used, each specific to a type of disease to be monitored. The system can detect when any of the targeted cells or diseases have been encountered, providing a general screen for a plurality of diseases. More specific tests can be used to follow up a positive result from the more general screen.

Analysis of the results of the measurement can comprise various techniques. A basic magnetic relaxometry measurement can provide information concerning the number of bound nanoparticles that were present in the measurement region by determining the magnetic moment of the particles exhibiting the characteristic Neel relaxation. The system can look for any measurement above zero if a sensitive detection is desired. If the targeting agent targets a substance that is normally present, then the system can look for a measurement above a threshold, e.g., a predetermined threshold (as in the common PSA test) or a determined threshold (as when looking for an increase or decrease in the presence of a targeted substance). The system can also look for patterns in the measurement, such as variations that might correlate with time of day or physical exertion. The system can also look for frequency of occurrence, e.g. if a cell is detected once per day as opposed to once per hour. The system can also look for measurement changes following occurrences such as chemotherapy, specific diet choices or changes, other medications or therapies.

The magnetic properties of the nanoparticles can also be used to urge the nanoparticles, and especially the nanoparticles that have bound to targeted substances, to particular regions. For example, an appropriate magnetic field can be used to encourage nanoparticles to remain in the measurement region for extended periods. As another example, an appropriate magnetic field can be used to urge nanoparticles to the portion of the body that is being measured, e.g., an arm if a wristwatch-like configuration is used.

Using the magnetic properties to encourage the presence of the targeted substance in a predetermined region of the body can improve the effective sensitivity of the magnetic relaxometry measurement. If can also be used to enrich the region with the targeted substance, so that a blood draw for immunohistochemistry or other analysis can be done in that region and the effective sensitivity of that procedure also improved.

The presence of nanoparticles on the targeted substance can also be beneficial after blood has been drawn. The targeted substance can be preferentially removed from the blood sample for further analysis, for example by magnetic separation as described in PCT/US2010/0514417 and U.S. Ser. No. 13/399,733 filed Feb. 17, 2012; each of which is incorporated herein by reference.

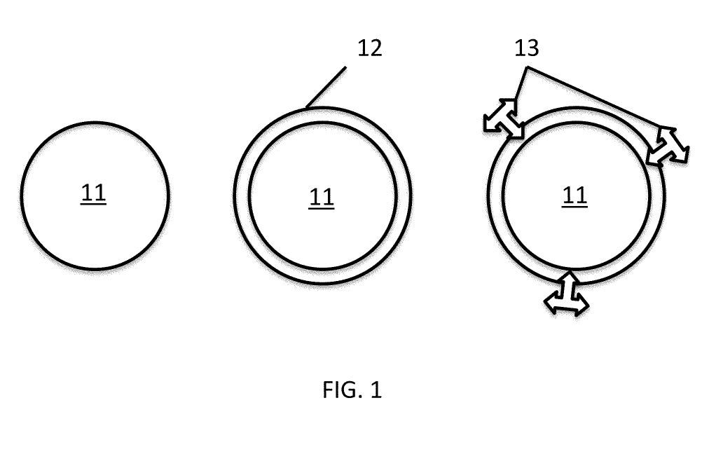

FIG. 1 is a schematic illustration of the preparation of targeted nanoparticles. At the left is an iron oxide nanoparticle core 11, such as those available from Senior Scientific LLC, and currently marketed under the PrecisionMRX trademark (PrecisionMRX is a trademark of Senior Scientific LLC). In the middle of the figure is a depiction of an iron oxide core 11 with a coating 12 that fosters stability in aqueous environments and that allows attachment of targeting agents. At the right is a depiction of the coated particle with targeting agents 13 attached, ready for injection into the subject.

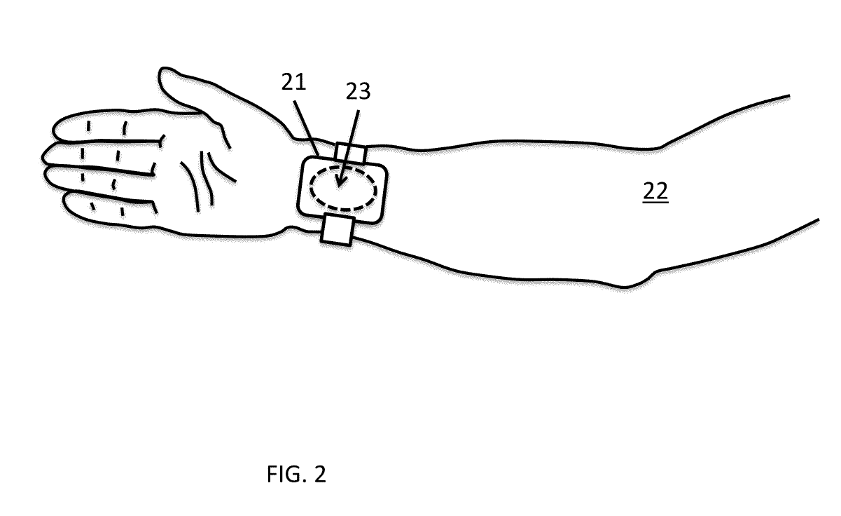

FIG. 2 is a schematic illustration of an example embodiment of the present invention deployed on the arm 22 of a subject. The magnetic relaxometry system 21 mounts in contact with the skin of the subject. The sensor portion of the system can mount in close proximity to the veins of the subject, to facilitate measurement of weak magnetic fields. Nanoparticles that have bound to targeted substances, when present in the measurement region 23, can be detected and measured by the system. The system can collect and analyze the data, or can communicate the date to a remote system (not shown) such as a computer or smart phone.

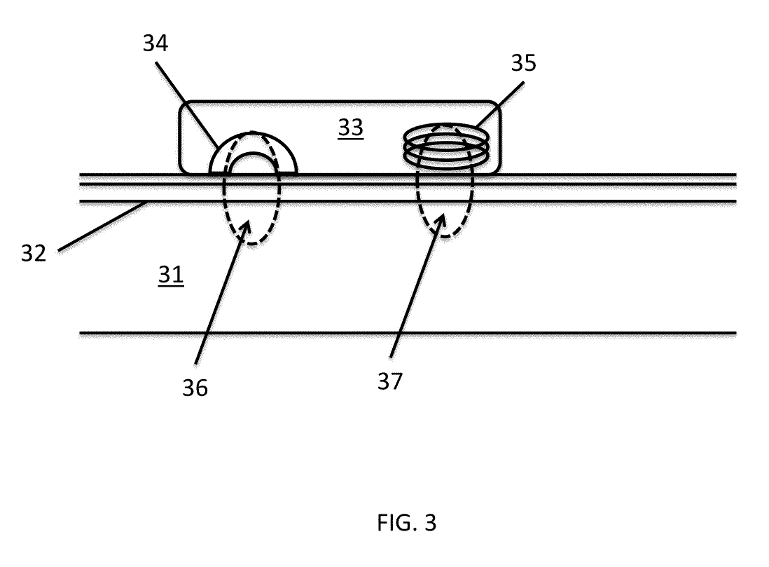

FIG. 3 is a schematic illustration of the placement of a magnetizing system and a sensor system in an example embodiment of the present invention. The subject is depicted with straight lines for ease of illustration. A portion of the subject is shown 32, e.g., an arm, leg, or finger of the subject. A blood-carrying element such as a vein or artery 32 is also depicted. A magnetic relaxometry system 33 is disposed next to the patient 31. The magnetic relaxometry system 33 comprises a magnetization system 35 and a sensing system 34. The magnetization system 35 magnetizes nanoparticles present in a magnetization region 37 of the patient. The sensing system 34 senses magnetic fields in a sensing region 36 of the patient. The magnetization system 35 and sensing system 34 are separated by a distance such that normal or expected travel of nanoparticles magnetized in the magnetization region 37 will position the nanoparticles in the sensing region 36 during the significant times in the magnetic relaxometry measurement. The separation shown in the figure is for illustration only; it is not to scale and the two regions can be closer together or farther apart, and can overlap in whole or in part. The magnetization system 35 can comprise any of a number of structures that impose a magnetic field one a region, such as various configurations of coils that carry electrical current to generate a magnetic field or permanent magnets. The sensing system can be any of various structures that sense low strength magnetic fields, such as sensors based on superconducting quantum interference devices and atomic magnetometers.

The present invention has been described in connection with various example embodiments. It will be understood that the above description is merely illustrative of the applications of the principles of the present invention, the scope of which is to be determined by the claims viewed in light of the specification. Other variants and modifications of the invention will be apparent to those of skill in the art.

We claim:

1. A method of determining the presence of a substance of interest in the circulatory system of a human or animal body, comprising:
(a) providing probes comprising nanoparticles conjugated with a targeting agent that preferentially binds with the substance of interest in the body;
(b) introducing the probes into the body;
(c) providing a magnetic relaxometry measurement system configured to detect nanoparticles within a measurement region of the system;
(d) positioning the magnetic relaxometry system relative to the body such that a portion of the circulatory system of the body is within the measurement region;
(e) using the magnetic relaxometry system to assess the presence of nanoparticles bound to the substance and within the measurement region;
(f) using the presence of nanoparticles to determine the presence of the substance in the circulatory system.

2. A method as in claim 1, wherein providing probes comprises providing superparamagnetic nanoparticles whose magnetization decays by Neel relaxation from saturated to about 0.1 of saturated in 5 seconds or less.

3. A method as in claim 2, wherein providing probes comprises providing nanoparticles having a biocompatible coating, and conjugated to an antibody that preferentially binds to one or more types of cells.

4. A method as in claim 3, wherein the one or more types of cells comprises one or more types of cancer cells.

5. A method as in claim 3, wherein the one or more types of cells comprises one or more types of cells correlated with the presence, growth or metastasis of cancer.

6. An apparatus for determining the presence of a substance of interest in the circulatory system of a human or animal body, comprising:
(a) A magnetization system, configured to impose a magnetic field upon a magnetization region comprising a portion of the circulatory system of a human or animal body;
(b) A magnetic measurement system, comprising one or more magnetic sensors configured to measure a magnetic field at a plurality of times in a measurement region comprising a portion of the circulatory system of a human or animal body;
(c) An analysis system, configured to determine the presence of superparamagnetic nanoparticles bound to one or more substances of interest from the magnetic field measurements and for the Neel relaxation properties of the nanoparticles.

7. An apparatus as in claim 6, wherein the magnetization system comprises one or more electrically conductive coils, and a power supply, wherein the power supply provides electrical current through the coils.

8. An apparatus as in claim 6, wherein the measurement system comprises one or more superconducting quantum interference devices.

9. An apparatus as in claim 6, wherein the measurement system comprises one or more atomic magnetometers.

10. An apparatus as in claim 6, wherein the magnetization system is configured to mount with an arm of a human body.

11. An apparatus as claim 6, wherein the measurement system is configured to mount with an arm of a human body.

12. An apparatus as in claim 10, wherein the measurement system is configured to mount with an arm of a human body.
